(19) United States
(12) Patent Application Publication
(10) Pub. No.: US 2024/0252253 A1
(43) Pub. Date: Aug. 1, 2024

(54) SYSTEMS, DEVICES, AND METHODS FOR MINIMALLY INVASIVE BRAIN SURGERY

(71) Applicant: Epica International, Inc., San Clemente, CA (US)

(72) Inventors: Gianluca Parrini, Pisa (IT); Luca Ferretti, Pisa (IT); Leonardo Manetti, Montevarchi (IT); Nicola Montemurro, Pisa (IT); Giusi Di Pietro, Pisa (IT)

(21) Appl. No.: 18/422,576

(22) Filed: Jan. 25, 2024

Related U.S. Application Data
(60) Provisional application No. 63/482,231, filed on Jan. 30, 2023.

(51) Int. Cl. A61B 34/20; A61B 17/00; A61B 17/02; A61B 17/34; A61B 34/00; A61B 34/32

(52) U.S. Cl. CPC A61B 34/20; A61B 17/0218; A61B 17/34; A61B 34/25; A61B 34/32; A61B 2017/00367; A61B 2017/3482; A61B 2034/2065; A61B 2034/252

(57) ABSTRACT

A brain surgery system having a robotic arm for guiding insertion of a retractor into a subject brain. The system includes an imaging system having a radiation source and a detector adapted to output imaging data based on the measured radiation. The system further includes a computer system having a processor and memory. A robotic arm is mounted on the imaging system and is controlled by software. An end effector is attached to the robotic arm and adapted to hold a retractor. The end effector includes a linear slide having a fixed portion adapted to attach to the robotic arm and a movable portion. The linear slide is adapted to move the retractor in a longitudinal direction of the retractor to insert the retractor into the subject brain. The end effector further includes a stand affixed to the linear slide and adapted to hold the retractor.

### CROSS-REFERENCE TO RELATED APPLICATION(S)

This application claims priority to U.S. Provisional Patent Application No. 63/482,231, filed Jan. 30, 2023, the contents of which are incorporated herein by reference.

### TECHNICAL FIELD

The present disclosure relates to systems, devices, and methods for minimally invasive brain surgery and, in particular, to brain surgery in which the surgeon is guided by a surgical navigation system which uses robotics and imaging.

### BACKGROUND

For brain surgery relating to hemorrhagic stroke or tumor removal it is common practice to use a device called “brain retractor.” This kind of device is composed of an external vial opened on both ends and an internal trocar. Proper placement of the retractor is of critical importance.

### SUMMARY

In one aspect, the disclosed embodiments provide a brain surgery system having a robotic arm for guiding insertion of a vial into a subject brain. The system includes an imaging system comprising a radiation source and a detector adapted to measure radiation emitted by the radiation source, the detector being further adapted to output imaging data based on the measured radiation. The system further includes a computer system having at least one processor and memory and a robotic arm mounted on the imaging system and controlled by software executing on the at least one processor. The system further includes an end effector attached to the robotic arm and adapted to hold a retractor. The end effector includes a linear slide having a fixed portion adapted to attach to the robotic arm and a movable portion, the linear slide being adapted to move the retractor in a longitudinal direction of the retractor to insert the retractor into the subject brain. The end effector further includes a stand affixed to the linear slide and adapted to hold the retractor.

Embodiments may include one or more of the following features, alone or in any feasible combination.

The imaging system may be a computed tomography (CT) imaging system. The radiation source may be adapted to emit X-rays. The computer system may be implemented as part of the imaging system. The computer system may be external to the imaging system and may be in communication with the imaging system via a direct connection and/or a network. The imaging data may be in Digital Imaging and Communications in Medicine (DICOM) format. The computer system may be adapted to receive the imaging data, to determine an insertion position and an insertion orientation, and to control the robotic arm to move the end effector to the insertion position and insertion orientation. The robot arm may be adapted to position and orient the end effector with at least five of freedom.

The linear slide may include a sensor to measure movement of the movable portion relative to the fixed portion. The end effector may include a knob turnable to move the movable portion relative to the fixed portion. The knob may be manually turnable or turnable via a motor. The knob may be turnable manually and via a motor. The end effector may include a lock adapted to fix a position of the movable portion relative to the fixed portion. The lock may include a second knob turnable to fix the position of the movable portion relative to the fixed portion. The fixed portion of the linear slide may include at least one rail, and the movable portion of the linear slide may include a bar in communication with the at least one rail and adapted to move along the at least one rail. The bar may be a substantially flat plate positioned between the at least one rail and a second rail. The bar may include two guides affixed to an underside thereof, the guides extending in a longitudinal direction of the bar and being positioned to engage with the at least one rail and the second rail, respectively.

The stand may be at a distal end of the movable portion of the linear slide and may extend in a direction substantially perpendicular to a longitudinal direction of the movable portion of the linear slide. The stand may include an elongate portion and a clamp portion adapted to hold the retractor in place. The clamp portion may include two opposing portions that together form a portion of a circle. The stand may further include a base portion affixed to the linear slide, with a receptacle to receive and lock in place the elongate portion of the stand.

The retractor may include a vial with a trocar positioned within an interior of the vial, the retractor being adapted to be inserted into a subject brain. The retractor may be adapted to allow removal of the trocar from the retractor while leaving the vial in place in the subject brain. The trocar may include, at a distal end thereof, a conical tip adapted to be inserted into the subject brain. The trocar may include, at a proximal end, a base portion adapted to be in communication with a corresponding base portion of the vial. The base portion of the trocar and the base portion of the vial may cooperate to prevent separation of the trocar and the vial as the retractor is inserted into and/or retracted from the subject brain.

The computer system may be adapted to execute software on the at least one processor to perform: displaying the imaging data on a display to provide one or more images of the subject brain; receiving a user input specifying a target location within a selected one of the one or more images of the subject brain; and receiving a user input specifying an insertion point of the retractor. The computer system may be further adapted to execute software on the at least one processor to perform: determining an insertion position and an insertion orientation based at least in part on the specified insertion point and the specified target location; and controlling the robotic arm to move an end effector to the insertion position and the insertion orientation, the end effector being attached to the robotic arm and holding the retractor. The system may further include a controller having a processor and being configured to control the robotic arm based on communication with the software executing on the at least one processor

In another aspect, the disclosed embodiments provide an end effector adapted to attach to a robotic arm and adapted to hold a retractor including a vial with a trocar positioned within an interior of the vial, the retractor being adapted to be inserted into a subject brain. The end effector includes a linear slide having a fixed portion adapted to attach to the robotic arm and a movable portion, the linear slide being adapted to move the retractor in a longitudinal direction of the retractor to insert the retractor into the subject brain. The end effector further includes a stand affixed to the linear slide and adapted to hold the retractor.

Embodiments may include one or more of the following features, alone or in any feasible combination.

The linear slide may include a sensor to measure movement of the movable portion relative to the fixed portion. The end effector may include a knob turnable to move the movable portion relative to the fixed portion. The knob may be manually turnable or turnable via a motor. The knob may be turnable manually and via a motor. The end effector may include a lock adapted to fix a position of the movable portion relative to the fixed portion. The lock may include a second knob turnable to fix the position of the movable portion relative to the fixed portion.

The fixed portion of the linear slide may include at least one rail, and the movable portion of the linear slide may include a bar in communication with the at least one rail and adapted to move along the at least one rail. The bar may be a substantially flat plate positioned between the at least one rail and a second rail. The bar may include two guides affixed to an underside thereof, the guides extending in a longitudinal direction of the bar and being positioned to engage with the at least one rail and the second rail, respectively. The stand may be at a distal end of the movable portion of the linear slide and may extend in a direction substantially perpendicular to a longitudinal direction of the movable portion of the linear slide. The stand may include an elongate portion and a clamp portion adapted to hold the retractor in place. The clamp portion may include two opposing portions that together form a portion of a circle. The stand may further include a base portion affixed to the linear slide, with a receptacle to receive and lock in place the elongate portion of the stand.

In another aspect, the disclosed embodiments provide a method for guiding insertion of a retractor into a brain using a brain surgery system comprising a robotic arm, an imaging system, and a computer system having at least one processor and memory, the imaging system comprising a radiation source and a detector adapted to measure radiation emitted by the radiation source, the detector being further adapted to output imaging data based on the measured radiation. The method receiving, by the detector, radiation emitted by the radiation source, including radiation that has passed through a subject brain positioned between the radiation source and the detector. The method further includes receiving, by the processor, imaging data output by the detector based on the receiving of the emitted radiation. The method further includes displaying the imaging data on a display to provide one or more images of the subject brain. The method further includes receiving a user input specifying a target location within a selected one of the one or more images of the subject brain. The method further includes receiving a user input specifying an insertion point of the retractor. The method further includes determining an insertion position and an insertion orientation based at least in part on the specified insertion point and the specified target location. The method further includes controlling the robotic arm to move an end effector to the insertion position and the insertion orientation, the end effector being attached to the robotic arm and holding the retractor. The method further includes moving, using a linear slide, the retractor in a longitudinal direction of the retractor to insert the retractor into the subject brain.

Embodiments may include one or more of the following features, alone or in any feasible combination.

The determining of the insertion position and the insertion orientation may result in, when the end effector may be at the insertion position: (i) the insertion orientation being aligned with an axis passing through the target location and the insertion point; and (ii) the retractor being spaced apart from the insertion point in an outward direction along the axis passing through the target location and the insertion point. The method may further include removing the trocar from the retractor while leaving the vial in place in the subject brain. The method may further include inserting one or more surgical tools into the vial to perform surgical operations in the subject brain. The surgical tools may include an aspirator or an endoscope. The one or more images of the subject brain may represent one or more computed cross-sections of the subject brain. The user input specifying the target location may be received via a graphical user interface displayed on the display. The user input specifying the target location may be received via a user input of location coordinates.

### BRIEF DESCRIPTION OF THE DRAWINGS

FIG. 1A depicts a brain surgery system having a robotic arm for guiding insertion of a retractor into a subject brain, according to disclosed embodiments.

FIG. 1B depicts the components of a retractor, including a vial and a trocar to be positioned inside the vial to form the assembled retractor, according to disclosed embodiments.

FIG. 1C depicts a retractor inserted into a subject brain, according to disclosed embodiments.

FIG. 1D depicts imaging of a subject brain and determined navigation geometries of the end effector, according to disclosed embodiments.

FIG. 2 is a block diagram of the brain surgery system, according to disclosed embodiments.

FIG. 3 depicts an end effector, which includes a linear slide adapted to attach to a robotic arm, according to disclosed embodiments.

FIG. 4 depicts the underside of the linear slide of the end effector, according to disclosed embodiments.

FIGS. 5A and 5B depict a stand affixed to the linear slide of the end effector and adapted to hold the retractor, according to disclosed embodiments.

FIG. 6 depicts the retractor, which may include a vial with a trocar positioned within an interior of the vial, the retractor being adapted to be inserted into a subject brain, according to disclosed embodiments.

FIG. 7 is a flowchart of a method for guiding insertion of a retractor into a subject brain using a brain surgery system having a robotic arm and an imaging system, according to disclosed embodiments.

Where considered appropriate, reference numerals may be repeated among the drawings to indicate corresponding or analogous elements. Moreover, some of the blocks depicted in the drawings may be combined into a single function.

### DETAILED DESCRIPTION

In the following detailed description, numerous specific details are set forth in order to provide a thorough understanding of embodiments of the invention. However, it will be understood by those of ordinary skill in the art that the embodiments of the present invention may be practiced without these specific details. In other instances, well-known methods, procedures, components, and circuits have not been described in detail so as not to obscure the present invention.

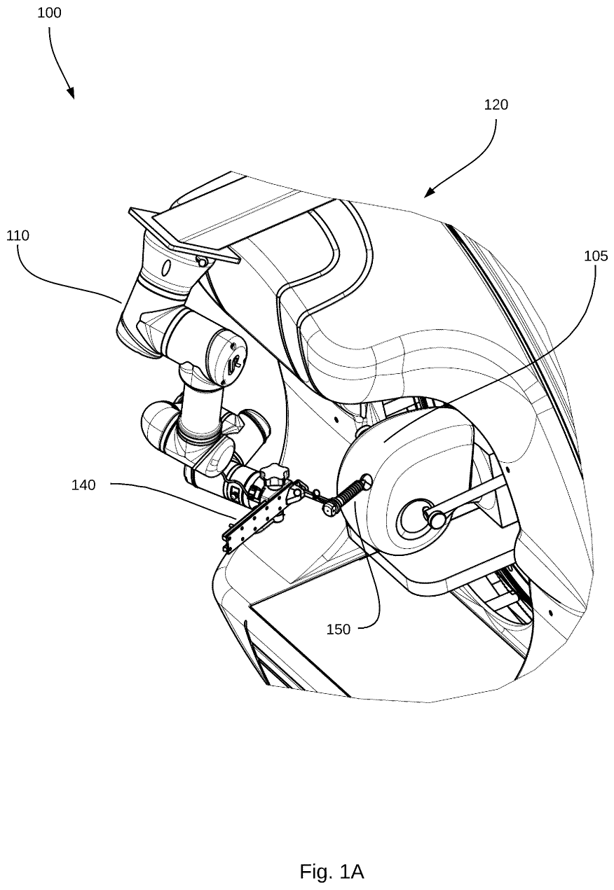

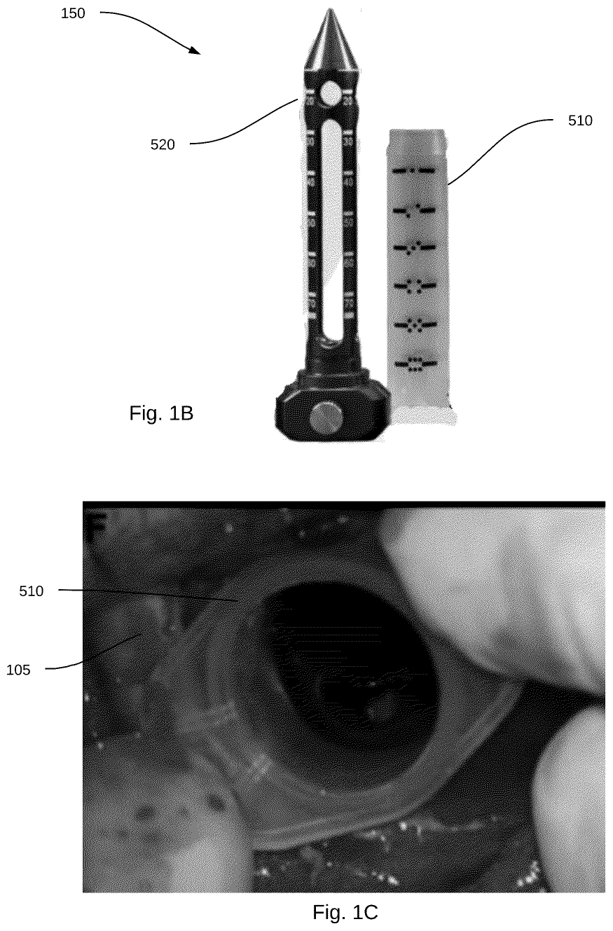

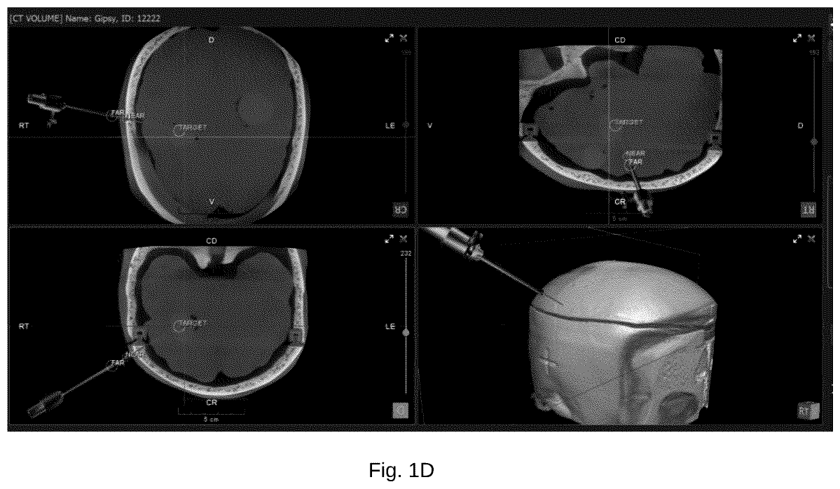

FIG. 1A depicts a brain surgery system 100 having a robotic arm 110 for guiding insertion of a retractor 150 into a subject brain 105. As discussed in further detail below, the system 100 allows a surgeon to perform minimally-invasive brain surgery by providing a navigation system and imaging in conjunction with specialized robotics-including a specialized end effector—for surgical applications such as brain hemorrhagic stroke, tumor removal, etc. The system 100 may be used in endoscopic brain tumor surgery (neuroendoscopy), which is a minimally invasive surgical procedure in which tumors can be removed through openings in the skull. In general, neuroendoscopy results in less pain and scarring and faster recovery than traditional surgery. The types of brain tumors which may be treated with neuroendoscopy include, e.g., pineal region tumors, pituitary tumors, Rathke's cleft cysts, skull base tumors, and ventricular tumors.

The brain surgery system 100 includes an imaging system 120. In embodiments, the imaging system 120 may be, for example, a Computed Tomograpy (CT) imaging system having a radiation source 122, e.g., an X-ray source, and a detector 125 adapted to measure radiation emitted by the radiation source 122. The detector 125 is adapted to output imaging data based on the measured radiation. In particular embodiments, the imaging data may be in Digital Imaging and Communications in Medicine (DICOM) format.

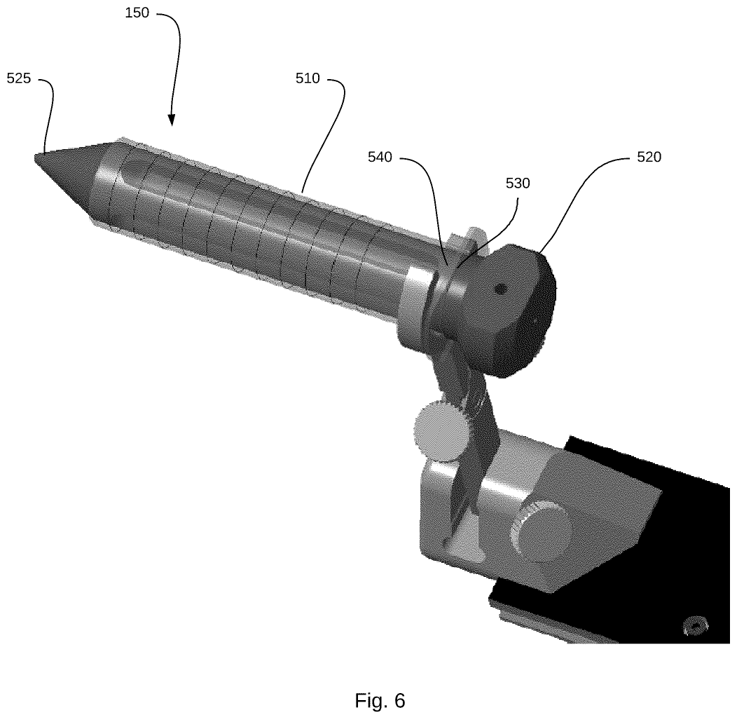

FIG. 1B depicts the components of a retractor 150, including a vial 510 and a trocar 520 to be positioned inside the vial to form the assembled retractor (see also FIG. 6). The tubular retractor essentially moves aside the folds and delicate tissues of the brain with less risk of damage than other surgical methods, i.e., it displaces the tissue instead of cutting through it. A tubular retractor can be especially useful in situations in which a tumor is located deep inside the brain. It also offers a less invasive option than traditional open surgery (i.e., craniotomy).

FIG. 1C depicts a vial 510 in an operational position following the insertion of the retractor 150 into the subject brain 105 and the subsequent removal of the retractor 150. In this position, the vial 510 allows a surgeon to enter the subject brain 105 through the interior of the vial 510, e.g., with a surgical tool, with minimally disruptive, navigable, trans-sulcal access through the subcortical space. In general, the retractor 150 displaces tissues of the subject brain 105, as opposed to disrupting them. Among other advantages, the system 100 allows a surgeon to accurately place the vial 510 in the subject brain 105 to minimize potential damage to the gray and white matter of the subject brain 105.

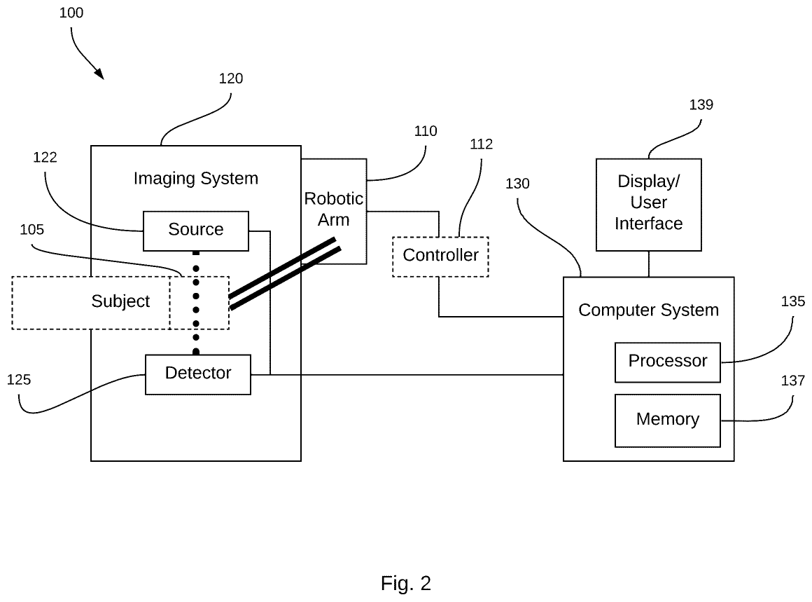

FIG. 2 is a block diagram of the brain surgery system 100. The system 100 includes an imaging system 120, as discussed above, with a computer system 130 having at least one processor 135, memory 137, and a display/user interface 139. In embodiments, the computer system 130 may be housed in, or otherwise implemented as part of, the imaging system 120. In particular embodiments, the computer system 130 may be a separate component or components, such as a standalone computer and computer display, in electronic communication with the imaging system 120, e.g., via a direct connection and/or a network.

Referring again to FIG. 1A, in embodiments, the robotic arm 110 is mounted on the imaging system 120 and is controlled by navigation software executing on the computer system 130 (see FIG. 2). For example, the robotic arm 110 may be a six-degree-of-freedom robotic arm. In embodiments, the robotic arm 110 may be part of a collaborative robotic system designed to be used without requiring protective measures by the users (e.g., surgeons and their assistants). An end effector 140 is adapted to attach to a robotic arm 110, e.g., a distal end of the robotic arm 110, and adapted to hold a retractor 150. In embodiments, the system 100 may include a robotic controller 112 having a processor and being configured to control the robotic arm 110 based on communication with the software executing the at least one processor 135 of the computer system 130.

FIG. 1D depicts imaging of a subject brain 105 and determined navigation geometries of the end effector 140, e.g., as presented by the display/user interface 139 of the computer system 130. In particular embodiments, the navigation software is adapted, i.e., designed, to receive and process the imaging data to determine an insertion position and an insertion orientation of the end effector 140. The navigation software also controls the robotic arm 110 to move the end effector 140 to the insertion position and insertion orientation.

In particular embodiments, there may be three relevant points used in defining insertion position and insertion orientation. A target location (or “target point”) may be specified by a user, e.g., by user input of location coordinates via a graphical user interface. For example, a surgeon may select a target location based on CT imaging presented via a user interface, e.g., as depicted in FIG. 1D. An insertion point (or “near point”) may be specified near to the skull surface where the retractor 150 will enter the skull to contact the subject brain 105. The target location and the insertion point define a straight line, i.e., an axis, as depicted in the figure. A “far point” may be defined which is spaced apart from the skull surface in an outward direction along the defined axis. The far point provides, in effect, a starting point for the insertion of the retractor 150 in the subject brain 105 through action of the end effector 140, as described in further detail below. Thus, the determination of the insertion position and the insertion orientation results in, when the end effector is at the insertion position: (i) the insertion orientation being aligned with an axis passing through the target location and the insertion point; and (ii) the retractor being spaced apart from the insertion point in an outward direction along the axis passing through the target location and the insertion point.

As noted above, the robot arm 110 may be adapted to position and orient the end effector 140 with six degrees of freedom-three dimensions for the position and three dimensions for the orientation. In embodiments, the end effector 140 may include a retractor 150 having a shape which is symmetric about a longitudinal axis thereof, e.g., cylindrical, in which case only five degrees of freedom may be needed, since it may not be necessary to define rotation about the longitudinal axis of the retractor 150. Nevertheless, a sixth degree of freedom may be used to define rotation about the longitudinal axis of the retractor 150 to rotate end parts of the robotic arm 110 about the longitudinal axis to better accommodate the robotic components within the operating scene.

Thus, a brain surgery system may include a computer system that is in communication with a radiological imaging system, a robotic arm, and a sensorized end effector. The computer system may be in communication with a display and graphical user interface. The computer system may include navigation software capable of calculating an insertion position and insertion orientation of the end effector, thereby allowing the operator to determine and set an insertion position. The computer system may instruct the robotic arm to move the end effector to align with the set insertion position and insertion orientation. Images from the radiological imaging system may be sent to the computer system and displayed, which may allow the surgeon to view the target area, e.g., lesion, to be treated. Using the images from the radiological imaging system, the navigation software may allow the surgeon to find the best axis of intervention point, i.e., insertion point.

In general, the surgeon may use the radiological images to localize the lesion and decide on a strategic and/or efficient path to reach the lesion. The path may be determined based at least in part on minimizing the depth of brain matter to pass through and/or minimizing the formation of additional lesions, which can result from inserting the retractor into the white/grey matter of the subject brain. In addition to the radiological images, any data from a patient monitoring or tracking device may also be sent to the computer system. The navigation software on the computer system may show the axis of intervention point and may determine or assist in determining the safest and most efficient axis of intervention point.

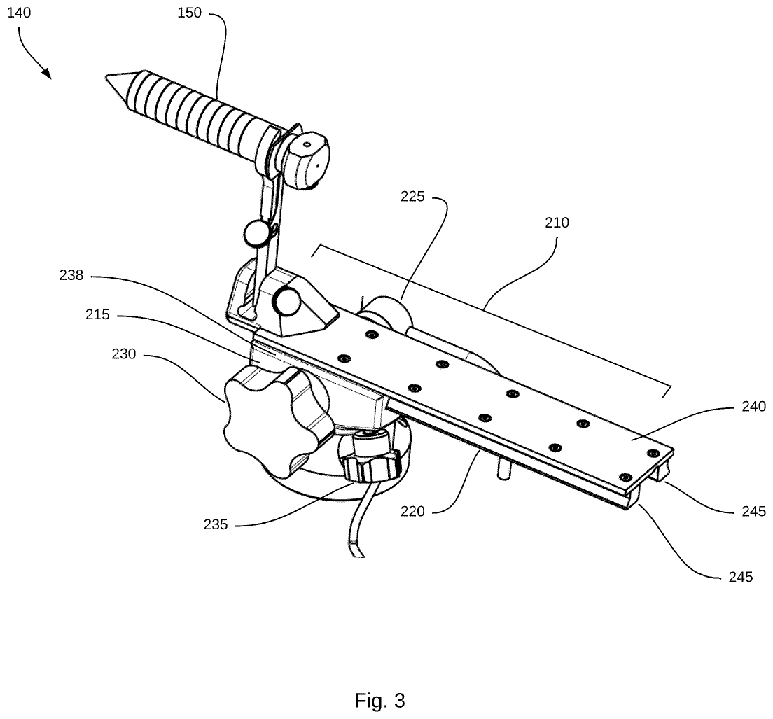

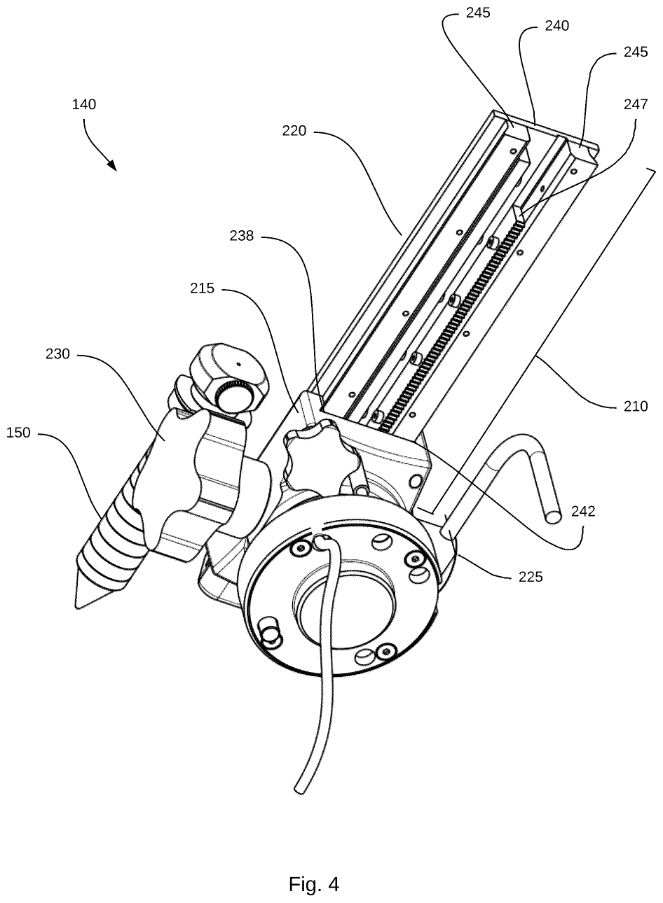

FIG. 3 depicts an embodiment of the end effector 140, which includes a linear slide 210 having a fixed portion 215 adapted to attach to the robotic arm 110 and a movable portion 220. The linear slide 210 is adapted to move the retractor 150 in a longitudinal direction of the retractor 150 so that the retractor 150 can be inserted into the subject brain in a substantially linear motion, in accordance with the shape and function of the vial 510. In particular embodiments, the linear slide 210 includes a sensor 225 to measure movement of the movable portion 220 relative to the fixed portion 215. A turnable knob 230 may be adapted to move the movable portion 220 relative to the fixed portion 215. For example, the knob 230 may be manually turnable, e.g., by a surgeon, and may have a gear (not shown) formed on a rear portion which interacts with other mechanical components, e.g., a toothed track 247 (see FIG. 4). In particular embodiments, the knob may be turnable manually, by a motor, or both. The end effector 140 may include a lock, e.g., a second knob 235, adapted to fix a position of the movable portion 220 of the linear slide 210 relative to the fixed portion 215.

FIG. 4 depicts the underside of the linear slide 210 of the end effector 140. In particular embodiments, the fixed portion 215 of the linear slide 210 includes a first rail 238, and the movable portion 220 of the linear slide 210 includes a bar 240 in communication with the first rail 238 and adapted to move along the first rail 238. The bar 240 may be a substantially flat plate positioned between the first rail 238 and a second rail 242. The bar 240 may include two guides 245 affixed to an underside thereof. The guides 245 may extend in a longitudinal direction of the bar 240 and may be positioned to engage with the first rail 238 and the second rail 242, respectively.

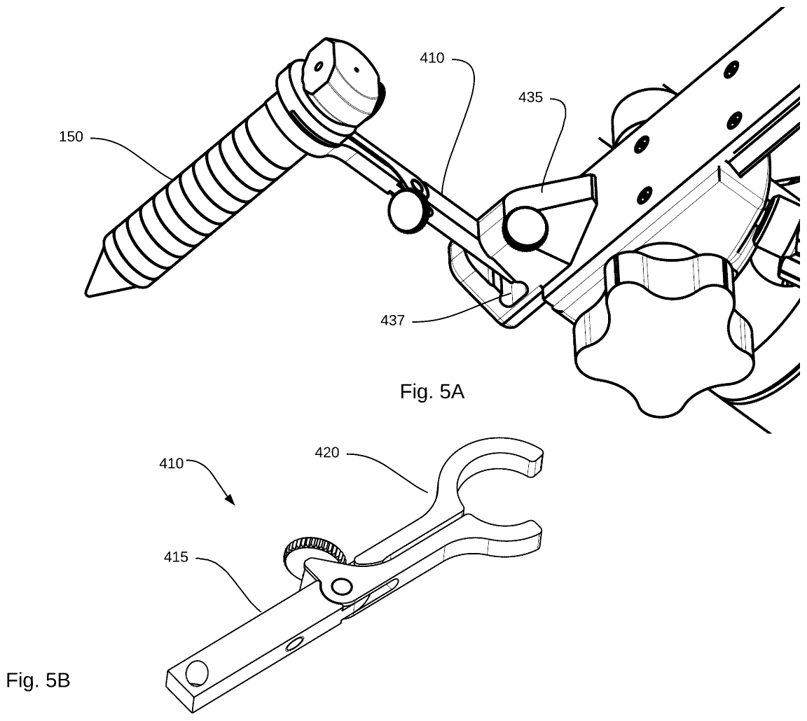

FIGS. 5A and 5B depict a stand 410 affixed to the linear slide 210 of the end effector 140 and adapted to hold the retractor 150. In particular embodiments, the stand 410 is at a distal end of the movable portion 220 of the linear slide 210 and extends in a direction substantially perpendicular to the movable portion 220 of the linear slide 210. In particular embodiments, the stand 410 may have an elongate portion 415 and a clamp portion 420, with the clamp portion 420 having two opposing portions that together form a portion of a circle. The clamp portion 420 may be adapted to clamp onto the retractor 150 to hold it in place. The stand 410 may further include a base portion 435 affixed to the linear slide 210, with a receptacle 437 to receive and lock in place the elongate portion 415 of the stand 410. The stand 410 may be implemented in various sizes based at least in part on corresponding sizes of the retractor 150 and, in particular, the size of the vial 510.

FIG. 6 depicts the retractor 150, which may include a vial 510 with a trocar 520 positioned within an interior of the vial 510, the retractor 150 being adapted to be inserted into a subject brain. In particular embodiments, the vial 510 may be hollow and substantially tube-shaped, e.g., cylindrical. The vial 510 may be formed of glass or other materials suitable for insertion into the human body and, in particular, the brain. The retractor 510 is adapted to allow removal of the trocar 510 from the retractor 510, while leaving the vial 510 in place in the subject brain.

The trocar 520 may include, at a distal end thereof, a conical tip 525 adapted to be inserted into the subject brain. In particular embodiments, the conical tip 525 of the trocar may be between about 10 mm and about 20 mm in length in a longitudinal direction of the retractor 150 or between about 14 mm and about 16 mm in length. The trocar 520 may be formed of titanium or other materials suitable for insertion into the human body and, in particular, the brain.

In particular embodiments, the trocar 520 may include, at a proximal end, a base portion 530 which is adapted to be in communication with a corresponding base portion 540 of the vial 510. The base portion 540 of the vial 510 may include a circumferential rim having a textured outer surface to allow the vial 510 to be more securely gripped. The base portion 530 of the trocar 520 and the base portion 540 of the vial 510 may cooperate to prevent separation of the trocar 520 and the vial 510 as the retractor 150 is inserted into and/or extracted from the subject brain 105. For example, an inner circumferential surface of the base portion 540 of the vial 510 may form an interference fit with the outer circumferential surface of the base portion 530 of the trocar 520 when the trocar 520 is inserted into the vial 510. In particular embodiments, part of the base portion 530 of the trocar 520 may be too wide to fit inside the vial 510 and may act as a stop as the trocar 520 is inserted into the vial 510.

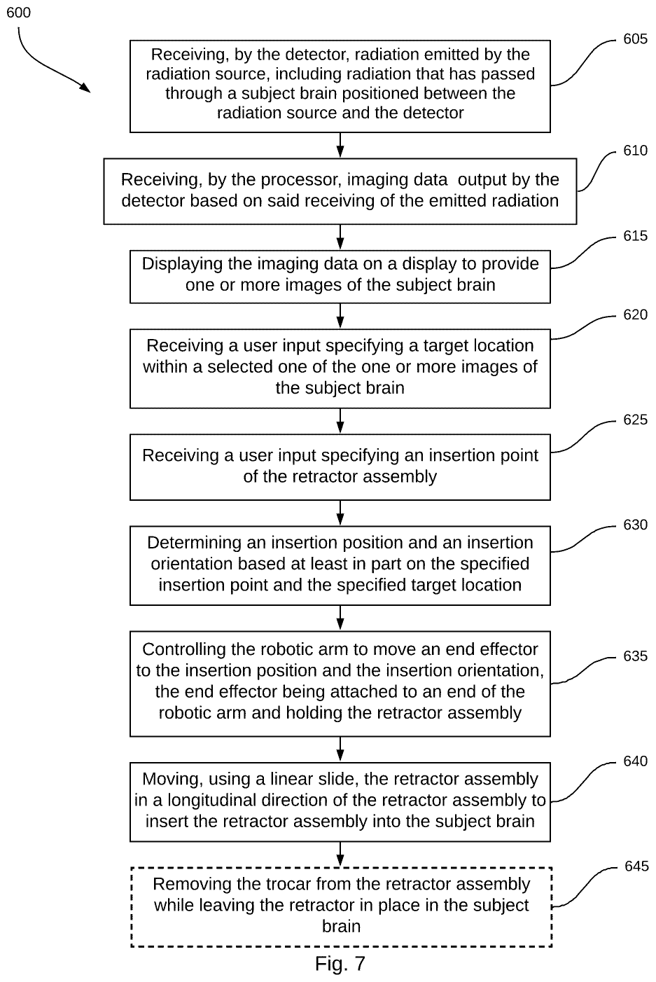

FIG. 7 is a flowchart of a method 600 for guiding insertion of a retractor into a subject brain using a brain surgery system having a robotic arm and an imaging system, such as, for example, the brain surgery system 100 discussed above. The method 600 includes receiving, by the detector, radiation emitted by the radiation source, including radiation that has passed through a subject brain positioned between the radiation source and the detector (605). The method 600 further includes receiving, by the processor, imaging data output by the detector based on the receiving of the emitted radiation (610).

The method 600 further includes displaying the imaging data on a display to provide one or more images of the subject brain (615). In particular embodiments, the images of the subject brain may represent one or more computed cross-sections of the subject brain. The method 600 further includes receiving a user input specifying a target location within a selected one of the one or more images of the subject brain (620). In particular embodiments, the user input specifying the target location may be received via a graphical user interface displayed on the display (see FIG. 1D). Furthermore, the user input specifying the target location may be received via a user input of location coordinates.

The method 600 further includes receiving a user input specifying an insertion point of the retractor (625). The method 600 further includes determining an insertion position and an insertion orientation based at least in part on the specified insertion point and the specified target location (630). The method 600 further includes controlling the robotic arm to move an end effector to the insertion position and the insertion orientation, the end effector being attached to the robotic arm and holding the retractor (635). In particular embodiments, the determining of the insertion position and the insertion orientation may result in, when the end effector is at the insertion position: (i) the insertion orientation being aligned with an axis passing through the target location and the insertion point; and (ii) the retractor being spaced apart from the insertion point in an outward direction along the axis passing through the target location and the insertion point.

The method 600 further includes moving, using a linear slide, the retractor in a longitudinal direction of the retractor to insert the retractor into the subject brain (640). In particular embodiments, the method 600 may further include removing the trocar from the retractor while leaving the vial in place in the subject brain (645). The method 600 may further include inserting one or more surgical tools into the vial to perform surgical operations in the subject brain. The surgical tools include such things as an aspirator, an endoscope, etc.

Aspects of the present invention may be embodied in the form of a system, a computer program product, or a method. Similarly, aspects of the present invention may be embodied as hardware, software, or a combination of both. Aspects of the present invention may be embodied as a computer program product saved on one or more computer-readable media in the form of computer-readable program code embodied thereon.

A computer-readable medium may be a computer-readable storage medium. A computer-readable storage medium may be, for example, an electronic, optical, magnetic, electromagnetic, infrared, or semiconductor system, apparatus, or device, or any combination thereof.

Computer program code in embodiments of the present invention may be written in any suitable programming language. The program code may execute on a single computer, or on a plurality of computers. The computer may include a processing unit in communication with a computer-usable medium, where the computer-usable medium contains a set of instructions, and where the processing unit is designed to carry out the set of instructions.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

1. A brain surgery system having a robotic arm for guiding insertion of a retractor into a subject brain, the system comprising:
an imaging system comprising a radiation source and a detector adapted to measure radiation emitted by the radiation source, the detector being further adapted to output imaging data based on the measured radiation;
a computer system having at least one processor and memory;
a robotic arm mounted on the imaging system and controlled by software executing on said at least one processor; and
an end effector attached to the robotic arm and adapted to hold a retractor, the end effector comprising:
a linear slide having a fixed portion adapted to attach to the robotic arm and a movable portion, the linear slide being adapted to move the retractor in a longitudinal direction of the retractor to insert the retractor into the subject brain; and
a stand affixed to the linear slide and adapted to hold the retractor.

2. The system of claim 1, wherein the computer system is adapted to receive the imaging data, to determine an insertion position and an insertion orientation, and to control the robotic arm to move the end effector to the insertion position and insertion orientation.

3. The system of claim 1, wherein the linear slide comprises a sensor to measure movement of the movable portion relative to the fixed portion.

4. The system of claim 1, wherein the end effector comprises a knob turnable to move the movable portion relative to the fixed portion.

5. The system of claim 1, wherein the fixed portion of the linear slide comprises at least one rail, and the movable portion of the linear slide comprises a bar in communication with said at least one rail and adapted to move along said at least one rail, wherein the bar is a substantially flat plate positioned between said at least one rail and a second rail.

6. The system of claim 5, wherein the bar comprises two guides affixed to an underside thereof, the guides extending in a longitudinal direction of the bar and being positioned to engage with said at least one rail and the second rail, respectively.

7. The system of claim 1, wherein the stand is at a distal end of the movable portion of the linear slide and extends in a direction substantially perpendicular to a longitudinal direction of the movable portion of the linear slide.

8. The system of claim 1, wherein the stand comprises an elongate portion and a clamp portion adapted to hold the retractor in place, wherein the stand further comprises a base portion affixed to the linear slide, with a receptacle to receive and lock in place the elongate portion of the stand.

9. The system of claim 1, wherein the retractor comprises a vial with a trocar positioned within an interior of the vial, the retractor being adapted to be inserted into a subject brain, wherein the retractor is adapted to allow removal of the trocar from the retractor while leaving the vial in place in the subject brain.

10. The system of claim 9, wherein the trocar comprises, at a proximal end, a base portion adapted to be in communication with a corresponding base portion of the vial, wherein the base portion of the trocar and the base portion of the vial cooperate to prevent separation of the trocar and the vial as the retractor is inserted into and/or retracted from the subject brain.

11. The system of claim 1, wherein the computer system is adapted to execute software on said at least one processor to perform:
displaying the imaging data on a display to provide one or more images of the subject brain;
receiving a user input specifying a target location within a selected one of said one or more images of the subject brain; and
receiving a user input specifying an insertion point of the retractor.

12. The system of claim 11, wherein the computer system is further adapted to execute software on said at least one processor to perform:
determining an insertion position and an insertion orientation based at least in part on the specified insertion point and the specified target location; and
controlling the robotic arm to move an end effector to the insertion position and the insertion orientation, the end effector being attached to the robotic arm and holding the retractor.

13. The system of claim 1, further comprising a controller having a processor and being configured to control the robotic arm based on communication with the software executing on said at least one processor.

14. An end effector adapted to attach to a robotic arm and adapted to hold a retractor including a vial with a trocar positioned within an interior of the vial, the retractor being adapted to be inserted into a subject brain, the end effector comprising:
a linear slide having a fixed portion adapted to attach to the robotic arm and a movable portion, the linear slide being adapted to move the retractor in a longitudinal direction of the retractor to insert the retractor into the subject brain; and
a stand affixed to the linear slide and adapted to hold the retractor.

15. The end effector of claim 14, wherein the linear slide comprises a sensor to measure movement of the movable portion relative to the fixed portion.

16. The end effector of claim 14, further comprising a knob turnable to move the movable portion relative to the fixed portion.

17. The end effector of claim 14, wherein the fixed portion of the linear slide comprises at least one rail, and the movable portion of the linear slide comprises a bar in communication with said at least one rail and adapted to move along said at least one rail, wherein the bar is a substantially flat plate positioned between said at least one rail and a second rail.

18. The end effector of claim 17, wherein the bar comprises two guides affixed to an underside thereof, the guides extending in a longitudinal direction of the bar and being positioned to engage with said at least one rail and the second rail, respectively.

19. The end effector of claim 14, wherein the stand is at a distal end of the movable portion of the linear slide and extends in a direction substantially perpendicular to a longitudinal direction of the movable portion of the linear slide.

20. The end effector of claim 14, wherein the stand comprises an elongate portion and a clamp portion adapted to hold the retractor in place, wherein the clamp portion comprises two opposing portions that together form a portion of a circle.

21. The end effector of claim 20, wherein the stand further comprises a base portion affixed to the linear slide, with a receptacle to receive and lock in place the elongate portion of the stand.

22. A method for guiding insertion of a retractor into a brain using a brain surgery system comprising a robotic arm, an imaging system, and a computer system having at least one processor and memory, the imaging system comprising a radiation source and a detector adapted to measure radiation emitted by the radiation source, the detector being further adapted to output imaging data based on the measured radiation, the method comprising:
receiving, by the detector, radiation emitted by the radiation source, including radiation that has passed through a subject brain positioned between the radiation source and the detector;
receiving, by the processor, imaging data output by the detector based on said receiving of the emitted radiation;
displaying the imaging data on a display to provide one or more images of the subject brain;
receiving a user input specifying a target location within a selected one of said one or more images of the subject brain;
receiving a user input specifying an insertion point of the retractor;
determining an insertion position and an insertion orientation based at least in part on the specified insertion point and the specified target location;
controlling the robotic arm to move an end effector to the insertion position and the insertion orientation, the end effector being attached to the robotic arm and holding the retractor;
moving, using a linear slide, the retractor in a longitudinal direction of the retractor to insert the retractor into the subject brain.

23. The method of claim 22, wherein said determining the insertion position and the insertion orientation results in, when the end effector is at the insertion position: (i) the insertion orientation being aligned with an axis passing through the target location and the insertion point; and (ii) the retractor being spaced apart from the insertion point in an outward direction along the axis passing through the target location and the insertion point.

24. The method of claim 22, further comprising removing the trocar from the retractor while leaving the vial in place in the subject brain and inserting one or more surgical tools into the vial to perform surgical operations in the subject brain.
